(19) United States
(12) Patent Application Publication
(10) Pub. No.: US 2006/0106437 A1
(43) Pub. Date: May 18, 2006

(54) Method for modifying or resetting the circadian cycle using short wavelength light

(76) Inventors: Charles A. Czeisler, Natick, MA (US); Steven W. Lockley, Somerville, MA (US); Richard E. Kronauer, Tucson, AZ (US); George C. Brainard, Haddonfield, NJ (US)

Correspondence Address: STERNE, KESSLER, GOLDSTEIN & FOX PLLC, 1100 NEW YORK AVENUE, N.W., WASHINGTON, DC, 20005, US

(21) Appl. No.: 11/113,356

(22) Filed: Apr. 25, 2005

Related U.S. Application Data
(63) Continuation of application No. 10/890,203, filed on Jul. 14, 2004, now abandoned.
(60) Provisional application No. 60/486,442, filed on Jul. 14, 2003.

(51) Int. Cl. A61N 5/06

(52) U.S. Cl. 607/88

(57) ABSTRACT

The present invention is a method for modifying the circadian cycle of a human subject to a desired state. The method includes the steps of assessing the present circadian cycle of the human subject, determining the characteristics of a desired circadian cycle, selecting an appropriate time during which to apply a stimulus of light to effect a desired modification of the present circadian cycle, and applying the light stimulus at the selected appropriate time to achieve the desired circadian cycle for the human subject. The stimulus of light comprises monochromatic short wavelength light (446-483 nm) or white light substantially comprising short wavelength light.

### CROSS-REFERENCE TO RELATED APPLICATIONS

This application claims the benefit of a prior-filed provisional application, having Provisional Application No. 60/486,442 filed on Jul. 14, 2003.

### STATEMENT REGARDING FEDERALLY-SPONSORED RESEARCH AND DEVELOPMENT

The present invention was made with government support under Grant No. R01-NS36590-05 awarded by the National Institutes of Health. The government has certain rights in the invention.

### BACKGROUND OF THE INVENTION

1. Field of the Invention

The present invention relates to a method for modifying or resetting the circadian cycle of a human subject. More particularly, the present invention relates to a method for modifying or resetting the circadian cycle of a human subject by applying a stimulus of light comprising monochromatic short wavelength light or white light substantially comprising short wavelength light.

2. Background Art

It is known that humans exhibit circadian rhythms or cycles in a variety of physiologic, cognitive and behavioral functions. Such cycles are driven by an internal biological clock or pacemaker that is located in the brain. It is also known that humans exhibit differing degrees of alertness or productivity during different “phases” of their circadian cycles.

Often, the activity and rest periods in which humans wish to engage do not coincide with the most appropriate phases of their circadian cycles. For instance, a transmeridian traveler experiences what is commonly referred to as “jet lag” because his or her circadian cycle is not “in tune” with the geophysical time of day of the destination location. In essence, the traveler's physiological clock (as based on the geophysical day of the departure location) lags or leads his or her desired activity-rest schedule, resulting in fatigue during the usual activity hours of the destination location and a sense of alertness or wakefulness during the usual rest hours of the destination location.

In a similar fashion, night-shift workers (such as factory workers, medical personnel, police and public utilities personnel) experience a desynchrony between the activities in which they wish to engage and their physiological ability to engage in such activities, as regulated by their circadian cycles. The misalignment between the phase of the worker's circadian cycle and scheduled night-work hours manifests itself as increased drowsiness during the early morning hours of 3:00 am to 7:00 am (assuming an habitual wake time of 7:00 am to 8:00 am). It is during this time frame that the circadian cycles of most humans are at their troughs or minimums, implying that they experience decreased alertness and fatigue and are, therefore, more prone to error or accident. Night-shift workers experience a corresponding difficulty in sleeping during the daytime hours after working at night, because the peak or maximum of the circadian cycle (when humans are most alert) is aligned with the hours allotted for sleep, as dictated by the night-shift worker's schedule. This results in sleep deprivation, which only decreases alertness and further increases the risk of error or accident on the part of the worker on subsequent night shifts. For workers in the medical field or for those who monitor processes in nuclear power plants, for example, such decreases in alertness could result in disastrous consequences.

There are various categories of sleep-related and affective disorders that are also believed to be related to misalignment between the circadian cycle and the desired activity-rest cycle. For example, the elderly often experience an advance in the phase of the circadian cycle to an earlier hour, which is manifested as sleepiness in the early evening hours of the day and an earlier than desired awakening during the morning hours of the day.

Other sleep-related disorders believed to be associated with misalignment of the circadian cycle to a desired activity-rest schedule include delayed-sleep phase insomnia, advanced sleep-phase insomnia, Seasonal Affective Disorder (SAD) and non-24-hour sleep-wake disorder.

It is known that light is the chief stimulus for regulating the circadian rhythms, seasonal cycles and neuroendocrine responses in many species, including humans, and that the durations of human melatonin secretion and sleep respond to changes in day length or photoperiod. Moreover, for decades clinical studies have shown that light therapy is effective for treating selected affective disorders, sleep problems and other disruptions of the circadian cycle. Thus, those skilled in the relevant scientific art realize that the circadian cycle may be phase-adjusted, modified or reset by exposing a human subject to an appropriately scheduled stimulus of light having select properties.

Methods for assessing and modifying the phase and amplitude of the circadian cycle are known. Several such methods are disclosed in U.S. Pat. No. 5,163,426 to Czeisler et al. for Assessment and Modification of a Subject's Endogenous Circadian Cycle; U.S. Pat. No. 5,167,228 to Czeisler et al. for Assessment and Modification of Circadian Phase and Amplitude; U.S. Pat. No. 5,176,133 to Czeisler et al. for Assessment and Modification of Circadian Phase and Amplitude; and U.S. Pat. No. 5,304,212 to Czeisler et al. for Assessment and Modification of a Human Subject's Circadian Cycle, collectively (the “Czeisler et al. patents”), the disclosures of which are incorporated herein, in their entirety, by reference.

The methods disclosed in the Czeisler et al. patents are premised on observations suggesting that a stimulus of bright light (ranging from 500-100,000 lux) has a direct effect on the circadian cycle, and that the strength of that direct-effect on the circadian cycle depends on the timing, intensity and duration of the stimulus of bright light.

U.S. Pat. No. 5,163,426 discloses a method for modifying a human subject's endogenous circadian cycle to a desired state, comprising the steps of assessing predefined specific characteristics of a present endogenous circadian cycle of the human subject, selecting one or more appropriate times in the present endogenous circadian cycle (based on the assessed characteristics) at which to apply a stimulus to effect a desired modification of the circadian cycle, and applying the stimulus, at the selected appropriate times in the present endogenous circadian cycle, to effect the desired modification of the circadian cycle, whereby the characteristics of the present endogenous circadian cycle are rapidly modified to substantially reduce the amplitude of the human subject's endogenous circadian cycle. The stimulus preferably comprises a pulse of bright light and may, optionally, comprise an episode of imposed darkness.

The assessing step of the above-described method comprises the steps of placing the subject in a semi-recumbent position, minimizing the subject's physical activity, feeding the subject small amounts of food at regular, closely-timed intervals, keeping the subject awake, measuring the characteristics of the present endogenous circadian cycle by measuring physiological parameters of the human subject (e.g., core body temperature, subjective alertness, melatonin secretion, urine volume, etc.), and forming a representation of the physiological parameters as a function of time. The described technique for assessing the phase and amplitude of the circadian cycle, both before and after application of a cycle-resetting or modifying stimulus regimen, and known as the “Constant Routine”, eliminates many of the confounding factors associated with assessment of the circadian phase. It forms a part of many existing methods and studies for assessing and modifying the circadian cycle, including the study and method of the present invention discussed in further detail below.

The Czeisler et al. patents also disclose a method for modifying a human subject's circadian cycle to a desired state comprising the steps of assessing the characteristics of the present circadian cycle of the subject and applying, at preselected times in the assessed present circadian cycle, pulses of bright light (and, optionally, pulses of darkness) of preselected duration, whereby the characteristics of the present endogenous circadian cycle are rapidly modified to the become the desired state of the human subject's circadian cycle. A mathematical model of the circadian pacemaker (having a forcing function), which takes the form of a second order differential equation of the van der Pol type, for use in assessing and modifying the circadian cycle of a human subject to a desired state is also taught in the Czeisler et al. patents.

The bright light stimulus for affecting modification of the circadian cycle to a desired state may also be defined in terms of “enhanced illumination” and “diminished illumination” and such methods are disclosed and claimed in U.S. Pat. No. 5,304,212.

U.S. Pat. No. 5,545,192 discloses that humans appear to sum circadian photic responses progressively, and that a human subject need not be exposed to light of a high intensity (e.g., 10,000 lux) for a long period of time (e.g., 5 hours) to evoke a shift in the circadian phase. In the subject patent, Czeisler et al. disclose that an increase in retinal light exposure requires a measurable duration of time to initiate the neurophysiological or neurohumoral chain of events responsible for mediating the circadian response to enhanced light exposure, and that such biological effects of enhanced light on the circadian pacemaker will persist on a diminishing trajectory for some duration of time following a reduction in the level of retinal light exposure. Thus, the circadian pacemaker appears to respond on a diminishing scale to the previous light stimulus even though an episode of darkness (or diminished light) follows exposure to enhanced light. Based on such a response, Czeisler et al. disclose that intermittent exposure to bright light can be as nearly effective as continuous exposure to bright light and put forth another method for modifying the circadian cycle of a human subject to a desired state. The method comprises the steps of applying an episode of intermittent light consisting of at least two pulses of enhanced-intensity light separated by at least one pulse of reduced-intensity light to the human subject. Approximately 20% of the duration of the episode of intermittent light comprises light of enhanced intensity. Like the other patents, Czeisler et al. disclose a mathematical model of the circadian pacemaker, which has been enhanced to reflect the findings that humans appear to sum circadian photic responses.

While it is true that bright light or light of an enhanced intensity (e.g., light ranging between 100 and 100,000 lux) has an effect on the circadian cycle, more recent research suggests that the circadian cycle receives photic input from photoreceptors not used for image-forming which are sensitive to specific wavelengths of light. More particularly, recent research reveals that the mammalian circadian pacemaker, situated in the hypothalamic suprachiasmatic nuclei (SCN), receives environmental photic input (perceived environmental light and dark cycles) from a specialized set of ganglion cells. The photic input entrains endogenous near. 24-hour rhythms (including pineal rhythms) to the environmental 24-hour light-dark cycle, to maintain appropriate phase relationships between rhythmic physiological and behavioral processes and periodic environmental factors. In addition to entraining pineal rhythms, light exposure can acutely suppress melatonin secretion. Acute, light-induced melatonin suppression, a broadly used indicator for photic input to the SCN, has been used to elucidate the ocular and neural physiology for circadian regulation.

The human circadian pacemaker is exquisitely sensitive to ocular light exposure, even in some people who are otherwise totally blind. Indeed, Czeisler and others have demonstrated light-induced melatonin suppression and circadian entrainment in humans with complete blindness and with specific color vision deficiencies. Taken together, such demonstrations suggest that melatonin regulation is controlled (at least in part, if not primarily) by photoreceptors that differ from known photoreceptors for vision or image-forming. Past studies have shown that the magnitude of the phase-resetting response to white light-depends on the timing, intensity, duration, number and patterns of exposure. Recent studies, however, show that exposure to monochromatic light of a particular wavelength (i.e., a short wavelength ranging between 446-483 nm or blue light) effects a phase delay and suppression of melatonin not heretofore expected or known, which indicates that, in humans, a particular photoreceptor may be primarily responsible for melatonin suppression and circadian phase shifting, having a peak absorbance distinct from that of the three-cone photopic system for vision or image-forming. Indeed, the peak sensitivity of the human circadian pacemaker to light appears to be blue-shifted relative to the three-cone visual photopic system, the sensitivity of which peaks at approximately 555 nm. The present invention seeks to account for the sensitivity of the circadian pacemaker to blue or short wavelength light by setting forth novel methods to shift the phase of the circadian cycle (i.e., phase-advance or phase-delay it) to reset or modify the circadian pacemaker.

### BRIEF SUMMARY OF THE INVENTION

The present invention seeks to incorporate the above findings to more effectively and efficiently modify the circadian cycle of a human subject to a desired circadian cycle or activity-rest schedule. In accordance with this objective, the present invention is a method for modifying the phase and amplitude of the human circadian cycle to a desired state comprising the steps of assessing the characteristics of the present circadian cycle, determining the characteristics of a desired circadian cycle, selecting an appropriate time with respect to the human subject's present circadian cycle during which to apply a light stimulus to effect a desired modification of the human subject's circadian cycle, where the light stimulus comprises light having a short wavelength, and applying the light stimulus at the selected appropriate time to modify the human subject's present circadian cycle to the desired state.

In another embodiment, the present invention is a method for modifying a human subject's circadian cycle to a desired state comprising the steps of determining the characteristics of a desired endogenous circadian cycle for the human subject, selecting an appropriate time with respect to the presumed phase of physiological markers of the human subject's present endogenous circadian cycle during which to apply a light stimulus to effect a desired modification of the present endogenous circadian cycle of the human subject, and applying the light stimulus at the selected time to achieve the desired endogenous circadian cycle for the human subject. The light stimulus comprises an episode of intermittent light consisting of at least two pulses of short wavelength light separated by at least one pulse of reduced light.

The findings and methods of the present invention can be utilized to modify the circadian cycles of shift workers or transmeridian travelers and those affected by sleep-related disorders or Seasonal Affective Disorder in a more effective and time/energy efficient manner.

### BRIEF DESCRIPTION OF THE DRAWINGS/FIGURES

The examples and methods of the present invention are best understood and appreciated by referring to the accompanying drawings in which:

FIG. 1 is a graphic representation of circadian phase delay shift after exposure to 460 nm and 555 nm monochromatic light; and

FIG. 2 is a graphic representation of individual melatonin profiles prior to, during and after exposure to 460 nm and 555 nm monochromatic light.

### DETAILED DESCRIPTION OF THE INVENTION

Several methods for assessing and modifying the circadian cycle of a human subject are disclosed and claimed in U.S. Pat. Nos. 5,163,426; 5,167,228 5,176,133, 5,304,212 and 5,545,192 to Czeisler et al., the disclosures of which are incorporated herein, in their entirety, by reference. While the circadian cycle of the human subject may be assessed and modified by the methods disclosed in the patents, the human circadian cycle may perhaps be more effectively and efficiently modified or reset by refining the methods to accommodate recent findings which indicate that a photoreceptor primarily responsible for melatonin suppression has a peak absorbance that is shifted to short wavelength light or blue light.

As noted above, the mammalian circadian oscillator, situated in the hypothalamic suprachiasmatic nuclei (SCN), receives environmental photic input from a specialized subset of photoreceptive retinal ganglion cells. Such photic information entrains endogenous near 24-hour rhythms to the environmental 24-hour light-dark cycle, to maintain appropriate phase relationships between rhythmic physiological and behavioral processes and periodic environmental factors. The human circadian pacemaker is exquisitely sensitive to ocular light exposure, even in some people who are otherwise totally blind. The magnitude of the resetting response to white light has been shown to depend on the timing, intensity, duration, number and pattern of exposures. Although wavelength-dependence as an inherent property of circadian photic phase-resetting was demonstrated over 40 years ago in single-celled algae in the seminal work of Hastings and Sweeney, it has not yet been systematically investigated in humans.

### EXAMPLE

Action spectra for non-image forming (visual) responses in humans have revealed a short-wavelength peak in spectral sensitivity (8ₘₐₓ 446-483 nm) for light-induced melatonin suppression and the latency of the cone-driven electroretinogram (ERG) b-wave following light adaptation. It is not known, however, whether similar spectral sensitivities exist for phase-shifts of the human circadian pacemaker. We therefore employed classical photobiological techniques to test the effects of monochromatic wavelengths on photic circadian phase-resetting in humans, as indicated by the timing of the pineal melatonin rhythm. Based on the relative efficacy of the melatonin suppression response, we hypothesized that monochromatic light having a wavelength of 460 nm would induce a greater phase shift compared to light exposure having a wavelength of 555 nm.

### Methods

We studied 16 healthy subjects (8 females and 8 males; mean age±SD=23.3±2.4 years; range 19-27 years) in the Intensive Physiology Monitoring Unit at the Brigham and Women's Hospital (BWH). The study was approved by the Human Research Committees at BWH and Thomas Jefferson University and subjects gave written informed consent prior to study. All had comprehensive physical, psychological and ophthalmological exams, including an Ishihara color blindness test. Subjects were studied for nine days in an environment free of time cues and circadian phase assessments were made by monitoring the melatonin secretory profile during two Constant Routines, heretofore described, before and after exposure to monochromatic light. Plasma was sampled every 30 minutes from Day 2, and every 20 minutes during monochromatic light exposure. For three subjects with incomplete plasma sampling, hourly salivary melatonin was substituted. Melatonin was assayed using direct radioimmunoassay (RIA) (ALPCO Diagnostics, NH). Plasma intra- and interassay coefficients of variation (CV) were <9% and <11%, respectively at 1.94 and 16.59 pg/ml. Saliva intra- and interassay CVs were <15% and <16%, respectively at 1.65 and 16.57 pg/ml.

Monochromatic light exposure of 6.5 hours was timed to start 9.25 hours before respective waketime during each subjects' baseline days, corresponding on average to approximately 6.75 hours before core body temperature minimum, a phase at which white light exposure induces robust phase delays. The monochromatic light stimulus was generated from a 1,200 W arc lamp, grating monochromator and a Ganzfeld exposure system (dome). See Brainard et al. (2001) Action spectrum for melatonin regulation in humans: Evidence for a novel circadian photoreceptor. J Neurosci 21:6405-6412. Spectral characteristics were confirmed using a PR-650 SpectraScan Colorimeter (CR-650, PhotoResearch Inc., CA).

For 1.5 hours prior to and during light exposure, subjects were seated and 15 minutes before exposure, a pupil dilator was administered after which time subjects wore black-out goggles until the light exposure. During light exposure, subjects were supervised continually and asked to maintain cycles of 90 minutes fixed gaze in the Ganzfeld dome and 10 minutes free gaze. Subjects were randomized for exposure to either 460 nm (8 subjects) or 555 nm (8 subjects) monochromatic light (+10 nm half-peak bandwidth) of equal photon density (2.8×10¹³ photons/cm²/s). Irradiances were measured with an IL1400 radiometer and SEL-033/F/W detector (International Light Inc., MA). During free gazes, eye level irradiance was approximately 1 μW/cm².

Phase shifts (mean±SD) were calculated as the difference in clock time between initial and final phase of the melatonin rhythm measuring during the first and second Constant Routines, respectively. Melatonin phase was defined as the dim light melatonin onset (DLMO) calculated from 25% of the fitted three-harmonic peak-to-trough amplitude (DLMO_{25%}) of the melatonin rhythm during the first Constant Routine. Melatonin suppression (mean±SD) was calculated from the difference in the area under the curve (AUC), calculated using the trapezoidal method, between the melatonin profiles during the light exposure compared to the corresponding clock times during the previous melatonin cycle on first Constant Routine. Significance was assessed using one-tailed Student's t-tests.

### Results

Circadian Phase-Resetting

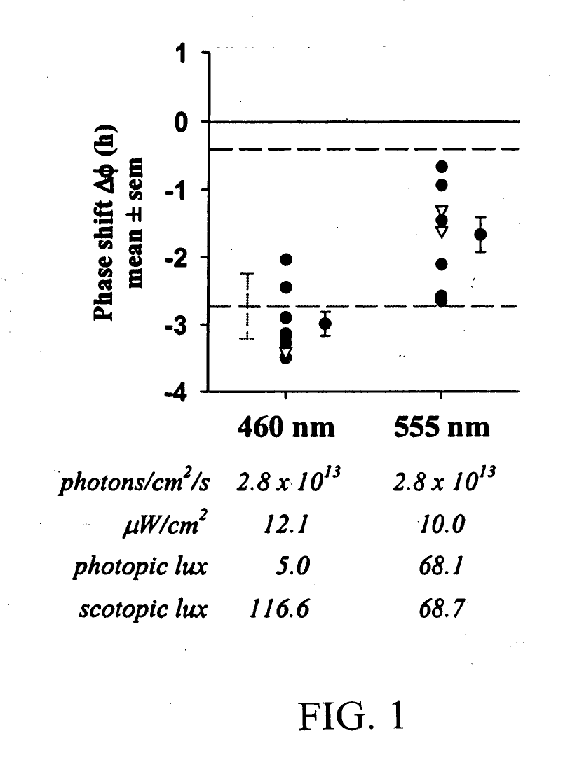

Monochromatic light exposure caused a phase delay of the melatonin rhythm in all subjects. FIG. 1 is a graphical representation of the phase delay shift of the plasma (X) or salivary ( [symbol] ) melatonin rhythm following exposure to 6.5 hours of monochromatic light having a wavelength of 460 nm or 555 nm. Delay shifts are negative by convention. The upper dashed line represents the average drift in phase due to circadian period. The lower dashed line shows the mean shift after 6.7 hours of exposure to approximately 10,000 lux of polychromatic white light at the same circadian phase in a similar study but without mydriasis (i.e., long-continued dilation of the pupil).

As shown in FIG. 1, exposure to 6.5 hours of 460 nm monochromatic light caused a significantly greater phase delay shift (−2.98±0.50 hour) than did exposure to 555 nm monochromatic light (−1.67±0.73 hour) (probability (p)<0.0006). When adjusted for the anticipated drift in phase due to circadian period (−0.4 hour if τ equals 24.2 hours), light having a wavelength of 460 nm caused twice as large a phase-shift as light having a wavelength of 555 nm light (−2.58 hours vs. −1.27 hours delay) despite equal photon densities of 2.8×10¹³ photons/cm²/s.

Melatonin Suppression

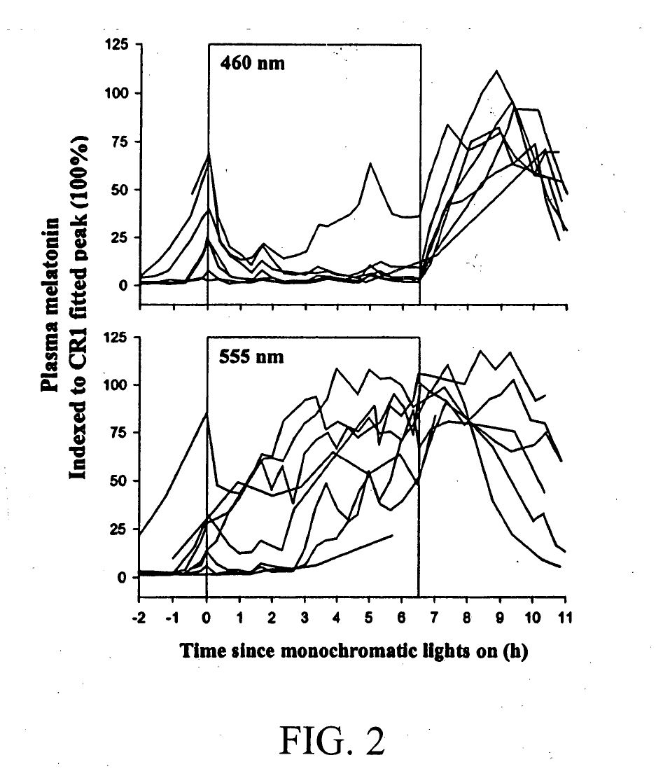

FIG. 2 is a graphical representation of individual melatonin profiles 2 hours prior to, during (boxed area) and 4 hours after 6.5 hours of exposure to monochromatic light, normalized to each individuals' fitted peak value during the first melatonin Constant Routine (25%=DLMO_{25%}). As shown in FIG. 2., all subjects exposed to 460 nm monochromatic light had at least a 65% suppression of the melatonin AUC during the 6.5-hour light exposure (range 65-96%). Suppression was more variable among subjects exposed to 555 nm monochromatic light (0-88%), including two individuals with no suppression of melatonin. On average, exposure to 6.5 hours of 460 nm monochromatic light caused a significantly greater suppression of melatonin (87.7±11.0%; 7 subjects) compared to that for 555 nm monochromatic light (39.1±34.1%; 8 subjects) (p=0.0021). The time course of the response also differed between the two groups. As indicated in the upper panel of FIG. 2, monochromatic light of 460 nm was able to suppress melatonin throughout the whole light exposure in all but one subject, who returned to the DLMO_{25%} level after 3.21 hours of exposure. Conversely, and as shown in the lower panel of FIG. 2, all but one subject exposed to 555 nm monochromatic light either failed to suppress to their DLMO_{25%} level at all or recovered to DLMO_{25%} after approximately 2.6 hours (3 subjects failed to suppress to their DLMO_{25%} level, while 4 subjects recovered to DLMO_{25%} after approximately 2.6 hours (at 0.46, 2.51, 3.16 and 4.19 hours)). As with polychromatic white light, the phase shift and suppression responses were highly correlated (correlation (r) 0.88, p<0.05).

### Discussion

The results of the example demonstrate that the efficacy of light in phase shifting human circadian rhythms is wavelength dependent and that the human circadian pacemaker is more sensitive to short (460 nm) versus long (555 nm) wavelengths of visible light. The photon fluxes (photons/cm2/s) of the two exposures do not correlate with the observed difference in the response following exposure to 460 nm and 555 nm monochromatic light (FIG. 1). Thus, the circadian photo-reception system does not simply count or average photons but rather is dependent on exposure to the particular wavelengths of energy. This blue-shift in sensitivity to visible light indicates that the photopic visual or image-forming system (i.e., bright light vision involving only the retinal cones) is not the primary photoreceptor system mediating phase-shifts of the endogenous circadian oscillator. Other cone-driven mechanisms that might weight the three cone inputs differently to that of color vision or some contribution from rods to the circadian entrainment process cannot, however, be ruled out. The photopic lux calculated for the two monochromatic exposures are negatively correlated with the magnitude of the phase shifts, demonstrating that the photopic visual system cannot be the primary mediator of the circadian phase shifting response, as revealed by the data graphically represented in FIG. 1. Furthermore, the relatively low scotopic lux provided in these monochromatic exposures, also shown in FIG. 1, make it unlikely that phase shifts of this magnitude can be accounted for solely by the visual scotopic system (dim light vision involving the retinal rods as photoreceptors), although this possibility cannot be excluded from the data. Although studies employing polychromatic light exposures have concluded that the photopic and/or the scotopic photoreceptor systems contribute to circadian resetting and melatonin suppression, they did not simultaneously employ monochromatic light exposures, equal photon densities, or control for circadian phase, thus confounding interpretation of those results.

The finding that the three-cone photopic system used for image-forming vision is not the primary mediator of circadian responses to light is consistent with previous studies of totally blind and red-green color-blind individuals (conducted by Czeisler et al. and Ruberg et al.), who maintain normal circadian phase shifting and melatonin suppression responses to white or green polychromatic light exposure. The results are also consistent with action spectra for non-circadian, non-image forming ocular responses in humans (8ₘₐₓ 446-483 nm) established by Brainard et al. (2001) which concluded that a novel non-classical visual photopigment may be the primary mediator of these responses. Although the above results are consistent with that hypothesis, they do not disprove an alternative photoreceptor mechanism. The above observations of an interaction between wavelength and duration of exposure on the time course of melatonin suppression over 6.5 hours may provide a tool to elucidate the photoreceptor(s) mediating this effect. Exposure to 460 nm light generated a prolonged, continuous signal that caused continual melatonin suppression for at least 6.5 hours. The eventual attenuation of melatonin suppression during exposure to 555 nm monochromatic light indicates that after several hours the suppressive drive was no longer adequate. Either the phototransduced signal at that wavelength and irradiance (10.0 ΦW/cm²) is declining or the pineal gland becomes less susceptible to suppression. At the photoreceptor level, there are at least two potential explanations of this finding: 1) a time- and wavelength-dependent change in sensitivity of a single (possibly novel) photoreceptor as a result of prolonged light exposure or 2) the involvement of two (or more) photoreceptor systems unequal in their ability to maintain a sustained response when exposed to constant monochromatic light.

The results are also consistent with parallel studies in nocturnal mammals with rod-dominated retinae, including visually impaired animals, which suggest that conventional rod- and/or cone-mediated photoreception used for sight is not required for non-image forming ocular responses, although not all studies concur. It has been proposed by others that these responses are mediated by an opsin, based on action spectra to behavioral responses. A novel opsin, melanopsin, is present in the majority of retinal ganglion cells that project to the SCN and is present in human retinae. These cells are directly photosensitive with a 8ₘₐₓ of 484 m in rats, close to that for pupillary reflexes in rod-dominated/rod-dominated coneless mice (8ₘₐₓ 479 nm), although wild type animals may be slightly more sensitive to longer wavelengths. Hence, melanopsin is a prime candidate for mediating circadian photoreception. Cryptochromes, flavoproteins used for detection of blue light in plants and lower organisms, including light detection for circadian responses, have also been proposed as circadian photopigments in mammals. Recent studies of knockout mice lacking melanopsin or cryptochrome have shown attenuation of circadian and pupillary reflex responses, although there is debate as to whether these potential photoreceptors are mutually redundant and whether rods and/or cones contribute to the responses observed in these animals. Whether or not such redundancy persists in intact wild-type animals and whether parallel systems exist in diurnal mammals, with differing visual photoreceptor systems, remains to be studied. Significant variations may also exist between diurnal and nocturnal mammals in the functional response of the SCN to direct retinal innervation, for example, in the proportion of cells that are excited or suppressed by direct photic input.

The results from the above example and other similar studies imply that shorter wavelength light may be more effective and energy-efficient compared to higher energy polychromatic white light for phase-shifting the human circadian pacemaker. The results may be applied to methods for treating circadian rhythm sleep disorders, or for quickly adapting the human circadian cycle to extreme or unusual photoperiods or to altered spectral environments. Exposure to the optimum balance of light wavelengths may also reduce the undesirable side-effects associated with therapeutic use of light exposure such as glare, visual discomfort, headaches and nausea.

The wider-ranging implication of the work is the demonstration that lux, the standard unit of illuminance used by the lighting industry and clinical research community, is inappropriate when assessing its effects on the circadian system or on melatonin suppression, as lux assumes that the light being measured has the same spectral (wavelength) distribution as the visual three-cone photopic system (λₘₐₓ 555 nm). The findings discussed above demonstrate this assumption to be inappropriate when relating photic drive to the magnitude of circadian resetting. Measurement and use of light to treat circadian rhythm sleep disorders should incorporate quantification of wavelength and irradiance in addition to the timing, number and pattern of exposures.

1. Methods and Mathematical Models Employing the Findings of the Example Regarding Short Wavelength Light

Akin to the previously referenced patents to Czeisler et al., the findings of the Example above may be integrated into the referenced methods and models for assessing and rapidly modifying the phase and amplitude of the endogenous circadian pacemaker, and for directly stimulating or inhibiting alertness and performance while awake. Indeed, it is envisioned that all of the methods of the Czeisler et al. patents, disclosed and incorporated herein in their entirety by reference, may be modified or refined to accommodate the recent findings that monochromatic short wavelength light (blue light) has an effect on melatonin suppression and, correspondingly, on the circadian cycle

Preferably, one method for modifying the phase and amplitude of the human circadian cycle to a desired state comprises the steps of (1) assessing the characteristics of the present circadian cycle, (2) determining the characteristics of a desired circadian cycle, (3) selecting an appropriate time with respect to the human subject's present circadian cycle during which to a light stimulus to effect a desired modification of the human subject's circadian cycle, and (4) applying the light stimulus at the selected appropriate time to modify the human subject's present circadian cycle to the desired state. The light stimulus is an episode or pulse of light having a relatively short wavelength of less than 500 nm, and is preferably monochromatic light having a wavelength of 446-483 nm. The light stimulus may optionally comprise an episode or pulse of imposed darkness. For the present invention, the episode or pulse of imposed darkness preferably comprises placing the human subject in a darkened room or exposing the human subject to reduced light of minimal intensity (e.g., less than 10 lux of white light), monochromatic light having a longer wavelength (greater than 600 nm), or polychromatic white light substantially comprising longer wavelength light.

It should be noted by those skilled in the art that a “pulse” or “episode” of short wavelength light may last for a brief or extended period of time, which may range from seconds or minutes to hours or days. The same holds true for an episode or pulse of imposed darkness depending on how the present circadian cycle of the human subject is to be modified. Moreover, an episode may comprise multiple pulses. In addition, each light stimulus regimen may be applied once or repeated over several hours or several days to effect a desired modification of the circadian cycle.

Like the methods disclosed in the Czeisler et al. patents referenced herein, assessment of the present circadian cycle and the timing for application of the light stimulus comprised of light having a short wavelength may be selected by referring to empirically derived or normative phase response data (which could be gathered from Constant Routine data that eliminates activity-related confounding factors associated with the sleep-rest cycle which otherwise mask the state of the endogenous circadian pacemaker) or by using a mathematical model in which the endogenous circadian pacemaker is a second order differential equation of the van der Pol type, transformed into two complementary first-order differential equations. For the present invention, which realizes and takes advantage of the effects of short wavelength light on the circadian cycle, the mathematical model takes the form of the “dynamic stimulus model” disclosed in Kronauer, R E, Forger D B, Jewett M E (1999), Quantifying human circadian pacemaker response to brief, extended and repeated light stimuli over the photopic range, J Biol Rhythms 14(6), 500-537, the disclosure of which is incorporated herein, in its entirety, by reference. The dynamic stimulus model (Process L) intervenes between the light stimuli and the traditional representation of the circadian pacemaker as a self-sustaining limit-cycle oscillator (Process P). The overall model incorporating Process L and Process P is intended to allow the prediction of phase shifts to photic stimuli of any temporal pattern (extended and brief light episodes) and any light intensity in the photopic range. Two time constants emerge in the Process L model: the characteristic duration for necessary pulses to achieve their full effect and the characteristic stimulus-free interval that can be tolerated without incurring an excessive penalty in phase shifting. The effect of reducing light intensity is incorporated in Process L as an extension of the time necessary for the light to be fully realized (a power-law relation between time and intensity). The referenced dynamic stimulus model can be used with monochromatic light of any wavelength or with light of any spectral composition, after defining a spectral sensitivity function, to mathematically model the circadian pacemaker and to assist in modification or resetting of the same.

Still another method for modifying a human subject's circadian cycle to a desired state comprises the steps of (1) determining the characteristics of a desired endogenous circadian cycle for the human subject, (2) selecting an appropriate time with respect to the presumed phase of physiological markers of the human subject's present endogenous circadian cycle during which to apply a light stimulus to effect a desired modification of the present endogenous circadian cycle of the human subject, and (3) applying the light stimulus at the selected appropriate time to achieve the desired endogenous circadian cycle for the subject. The light stimulus comprises an episode of intermittent light consisting of at least two pulses of short wavelength light separated by at least one pulse of reduced light. The short wavelength light has a wavelength less than 500 nm, and is preferably monochromatic light of 446-483 nm, while the reduced light is light of minimal intensity (e.g., less than 10 lux of white light), monochromatic light having a longer wavelength (greater than 600 nm), or polychromatic white light substantially comprising longer wavelength light.

It should be realized by those skilled in the art that depending on the application, a light stimulus of a particular short wavelength (i.e., blue light) may not be desirable for performing everyday tasks while simultaneously attempting to adapt the circadian cycle to a desired activity-rest schedule (e.g., the light may not be bright enough or the color may be inappropriate). For this reason, it is envisioned that the light stimulus of the methods of the present invention may also comprise polychromatic white light (which is visually more satisfying and appropriate) consisting substantially of short wavelength light (or other wavelengths of light appropriate for modifying the circadian phase). Moreover, it should be appreciated that the light administered to the human subject need not be limited to the preferred blue wavelength light, but could consist, on balance, of light having a wavelength capable of effecting melatonin suppression and shifting of the phase of the circadian cycle.

It is further envisioned that during times when light is required but suppression of melatonin secretion or phase-shifting is undesired or inappropriate, light comprised of a longer wavelength may be employed. For example, when biological functions (such as the need to urinate) disrupt sleep, a light source that emits longer wavelength light (e.g., a yellow, orange or red light) can be utilized to provide enough light to attend to the biological function, but avoid suppression of melatonin secretion. Methods that encompass the use of 1) short wavelength light to suppress melatonin secretion and shift the circadian phase and 2) longer wavelength light to stimulate melatonin secretion, are within the scope and spirit of the present invention. The present invention also contemplates the use of longer wavelength light (yellow, orange or red wavelength light) to safeguard against phase shifting or to maintain the phase of an existing circadian cycle.

2. Applications Utilizing the Methods and Mathematical Models Based on Exposure to Short Wavelength Light

The methods and findings of the present invention (which indicate that exposure to short wavelength (blue) light has a direct effect on suppression of melatonin and, correspondingly, an effect on the circadian cycle) may be applied to human subjects to treat jet lag, difficulties in adapting to night-shift work, phase-delayed or phase-advanced sleep disorders, and/or Seasonal Affective Disorder.

In general, light ranging from 2,000 lux to 12,000 lux has been used to modify the circadian cycle to treat Seasonal Affective Disorder, sleep disorders, non-24-hour sleep-wake disorders and other circadian disruptions. While these light levels appear to be therapeutically effective, many subjects complain that they produce side effects of visual glare, visual fatigue, eye pain and headaches. In addition, the devices that generate such levels of light require a substantial amount of energy and take some time to effect the desired change in the circadian cycle.

By treating circadian disruptions and disorders in accordance with the methods utilizing the short wavelength light discussed herein, wavelength emissions of the therapeutic equipment can be optimized, thereby reducing overall illuminances and avoiding the side effects and complaints mentioned above.

Modern industrialized societies use lights in homes, educational institutions, work places and public facilities to support visual performance, visual comfort, and aesthetic appreciation within a related enviromment. Given that light acts as a powerful regulator of the human circadian system, the methods of the present invention can be employed to provide illumination for human visual responses, as well as for circadian responses. As discussed herein, the findings suggest that humans have separate photoreceptors for visual and circadian responses to light. Thus, the present invention offers new approaches to therapeutic, as well as architectural lighting, to optimally stimulate both the visual system (by light of a specific intensity or illuminance) and the circadian or melatonin suppression system (by light having a specific (i.e., short) wavelength) in an effective and time/energy efficient manner.

It is envisioned that lights or lighting schemes based on the findings of the present disclosure can be developed and employed in the workplace to help a shift worker adapt to a night-shift work schedule and a corresponding rest schedule, by application of short wavelength light. Obviously, such lights must be configured to satisfy the requirement of the visual or image-forming photopic system, and for this purpose it may be desirable for the workplace to employ rooms having lights of differing wavelengths at different times, or polychromatic white light substantially comprised of light having a short wavelength light. A similar lighting plan could be employed on transmeridian flights to avoid jet lag or other sleep disruptions or in devices for treating other sleep related or affective disorders.

3. Devices

Naturally, any of the devices disclosed in the referenced Czeisler et al. patents incorporated herein in their entirety, by reference, such as ceiling lights, a free-standing lamp, hat, visor or light box, may be fitted with lamps which emit light of a short wavelength or lamps which emit polychromatic white light having the appropriate proportion of short wavelength light.

A device for generating the preferred short wavelength light of the present invention may be a specially designed arc lamp or it may be produced using an appropriate spectral filter.

Still other devices and methods for applying monochromatic light of 446-483 nm (or other wavelengths of light) are disclosed in International Publication No. WO/02/20079 A1 and U.S. Patent Application Publication No. US 2001/0056293 A1, the disclosures of which are incorporated herein, in their entirety, by reference. It is envisioned that such devices may be employed to practice the methods of the present invention to effect a modification or resetting of the circadian phase.

Regardless of the devices or application, the methods and findings of the present invention based on the effectiveness of short wavelength light to reset the circadian phase or modify the circadian cycle can be employed to effectively and time/energy efficiently assess the modification capacity of or to modify the circadian cycle of a human subject to a desired state.

What is claimed is:

1. A method for modifying the phase and amplitude of the human circadian cycle to a desired state comprising the steps of:
(a) assessing the characteristics of the present circadian cycle,
(b) determining the characteristics of a desired circadian cycle,
(c) selecting an appropriate time with respect to the human subject's present circadian cycle during which to apply a light stimulus to effect a desired modification of the human subject's circadian cycle, whereby said light stimulus comprises light having a short wavelength, and
(d) applying the light stimulus at the selected appropriate time to modify the human subject's present circadian cycle to the desired state.

2. The method of claim 1, wherein the wavelength of said short wavelength light ranges from 446 nm to 483 nm.

3. A method for modifying a human subject's circadian cycle to a desired state comprising the steps of:
(a) determining the characteristics of a desired endogenous circadian cycle for the human subject,
(b) selecting an appropriate time with respect to the presumed phase of physiological markers of the human subject's present endogenous circadian cycle during which to apply a light stimulus to effect a desired modification of the present endogenous circadian cycle of the human subject, said light stimulus comprising an episode of intermittent light consisting of at least two pulses of short wavelength light separated by at least one pulse of reduced light, and
(c) applying said light stimulus at said selected time to achieve said desired endogenous circadian cycle for said human subject.

4. The method of claim 3, wherein the wavelength of said short wavelength light is less than 500 nm.
